(19) United States
(12) Patent Application Publication
(10) Pub. No.: US 2005/0271684 A1
(43) Pub. Date: Dec. 8, 2005

(54) Apparatus and method for transdermal delivery of multiple vaccines

(76) Inventors: Joseph C. Trautman, Sunnyvale, CA (US); Peter E. Daddona, Menlo Park, CA (US); Michel J.N. Cormier, Mountain View, CA (US)

Correspondence Address: Ralph C. Francis;Francis Law Group, 1942 Embarcadero, Oakland, CA, 94606, US

(21) Appl. No.: 11/084,635

(22) Filed: Mar. 18, 2005

Related U.S. Application Data
(60) Provisional application No. 60/561,953, filed on Apr. 13, 2004.

(51) Int. Cl. C12Q 1/70; A61K 39/12; A61K 39/02; A61M 31/00

(52) U.S. Cl. 424/204.1; 604/500; 424/234.1; 514/291

(57) ABSTRACT

An apparatus and method for transdermally delivering an immunologically active agent comprising a delivery system having a microprojection array that includes a plurality of microprojections that are adapted to pierce through the stratum corneum into the underlying epidermis layer, or epidermis and dermis layers, the microprojection array having a plurality of array regions, each of the array regions having a different biocompatible coating disposed thereon, wherein at least one of the array region coatings includes an immunologically active agent. In one embodiment, each coating on the array regions includes a different immunologically active agent. In another embodiment, the biocompatible coating on a first array region includes an immunologically active agent and the biocompatible coating on a second array region includes an immune response augmenting adjuvant.

### CROSS-REFERENCE TO RELATED APPLICATIONS

This application claims the benefit of U.S. Provisional Application No. 60/561,953, filed Apr. 13, 2004.

### FIELD OF THE PRESENT INVENTION

The present invention relates generally to transdermal agent delivery systems and methods. More particularly, the invention relates to an apparatus, method and formulation for transdermal delivery of multiple vaccines.

### BACKGROUND OF THE INVENTION

Active agents (or drugs) are most conventionally administered either orally or by injection. Unfortunately, many active agents are completely ineffective or have radically reduced efficacy when orally administered, since they either are not absorbed or are adversely affected before entering the bloodstream and thus do not possess the desired activity. On the other hand, the direct injection of the agent into the bloodstream, while assuring no modification of the agent during administration, is a difficult, inconvenient, painful and uncomfortable procedure which sometimes results in poor patient compliance.

Hence, in principle, transdermal delivery provides for a method of administering active agents that would otherwise need to be delivered via hypodermic injection or intravenous infusion. The word “transdermal”, as used herein, is generic term that refers to delivery of an active agent (e.g., a therapeutic agent, such as a drug or an immunologically active agent, such as a vaccine) through the skin to the local tissue or systemic circulatory system without substantial cutting or penetration of the skin, such as cutting with a surgical knife or piercing the skin with a hypodermic needle. Transdermal agent delivery includes delivery via passive diffusion as well as delivery based upon external energy sources, such as electricity (e.g., iontophoresis) and ultrasound (e.g., phonophoresis).

Passive transdermal agent delivery systems, which are more common, typically include a drug reservoir that contains a high concentration of an active agent. The reservoir is adapted to contact the skin, which enables the agent to diffuse through the skin and into the body tissues or bloodstream of a patient.

As is well known in the art, the transdermal drug flux is dependent upon the condition of the skin, the size and physical/chemical properties of the drug molecule, and the concentration gradient across the skin. Because of the low permeability of the skin to many drugs, transdermal delivery has had limited applications. This low permeability is attributed primarily to the stratum corneum, the outermost skin layer which consists of flat, dead cells filled with keratin fibers (i.e., keratinocytes) surrounded by lipid bilayers. This highly-ordered structure of the lipid bilayers confers a relatively impermeable character to the stratum corneum.

As is well known in the art, skin is not only a physical barrier that shields the body from external hazards, but is also an integral part of the immune system. The immune function of the skin arises from a collection of residential cellular and humeral constituents of the viable epidermis and dermis with both innate and acquired immune functions, collectively known as the skin immune system.

One of the most important components of the skin immune system are the Langerhan's cells (LC), which are specialized antigen presenting cells found in the viable epidermis. LC's form a semi-continuous network in the viable epidermis due to the extensive branching of their dendrites between the surrounding cells. The normal function of the LC's is to detect, capture and present antigens to evoke an immune response to invading pathogens. LC's perform his function by internalizing epicutaneous antigens, trafficking to regional skin-draining lymph nodes, and presenting processed antigens to T cells.

The effectiveness of the skin immune system is responsible for the success and safety of vaccination strategies that have been targeted to the skin. Vaccination with a live-attenuated smallpox vaccine by skin scarification has successfully led to global eradication of the deadly small pox disease. Intradermal injection using ⅕ to 1/10 of the standard IM doses of various vaccines has been effective in inducing immune responses with a number of vaccines while a low-dose rabies vaccine has been commercially licensed for intradermal application.

It is, however, well known that many vaccine formulations are incompatible from a physicochemical standpoint. In order to administer these vaccines, they must be mixed at the time of injection or delivered via hypodermic injection.

As an alternative, transdermal delivery provides for a method of administering biologically active agents, particularly vaccines, that would otherwise need to be delivered via hypodermic injection, intravenous infusion or orally. Transdermal delivery offers improvements in both of these areas. Transdermal delivery, when compared to oral delivery, avoids the harsh environment of the digestive tract, bypasses gastrointestinal drug metabolism, reduces first-pass effects, and avoids the possible deactivation by digestive and liver enzymes. The digestive tract is also not subjected to the vaccine during transdermal administration. However, in many instances, the rate of delivery or flux of many biologically active agents via the traditional passive transdermal route is too limited to be immunologically effective.

One common method of increasing the passive transdermal diffusional agent flux involves pre-treating the skin with, or co-delivering with the agent, a skin permeation enhancer. A permeation enhancer, when applied to a body surface through which the agent is delivered, enhances the flux of the agent therethrough. However, the efficacy of these methods in enhancing transdermal protein flux has been limited, at least for the larger proteins, due to their size.

There also have been many techniques and systems developed to mechanically penetrate or disrupt the outermost skin layers thereby creating pathways into the skin in order to enhance the amount of agent being transdermally delivered. Early vaccination devices, known as scarifiers, generally include a plurality of tines or needles that were applied to the skin to and scratch or make small cuts in the area of application. The vaccine was applied either topically on the skin, such as disclosed in U.S. Pat. No. 5,487,726, or as a wetted liquid applied to the scarifier tines, such as, disclosed in U.S. Pat. Nos. 4,453,926, 4,109,655, and 3,136,314.

Scarifiers have been suggested for intradermal vaccine delivery, in part, because only very small amounts of the vaccine need to be delivered into the skin to be effective in immunizing the patient. Further, the amount of vaccine delivered is not particularly critical since an excess amount also achieves satisfactory immunization.

However, a serious disadvantage in using a scarifier to deliver an active agent, such as a vaccine, is the difficulty in determining the transdermal agent flux and the resulting dosage delivered. Also, due to the elastic, deforming and resilient nature of skin to deflect and resist puncturing, the tiny piercing elements often do not uniformly penetrate the skin and/or are wiped free of a liquid coating of an agent upon skin penetration.

Additionally, due to the self-healing process of the skin, the punctures or slits made in the skin tend to close up after removal of the piercing elements from the stratum corneum. Thus, the elastic nature of the skin acts to remove the active agent liquid coating that has been applied to the tiny piercing elements upon penetration of these elements into the skin. Furthermore, the tiny slits formed by the piercing elements heal quickly after removal of the device, thus limiting the passage of the liquid agent solution through the passageways created by the piercing elements and in turn limiting the transdermal flux of such devices.

Other systems and apparatus that employ tiny skin piercing elements to enhance transdermal agent delivery are disclosed in U.S. Pat. Nos. 5,879,326, 3,814,097, 5,279,54, 5,250,023, 3,964,482, Reissue U.S. Pat. No. 25,637, and PCT Publication Nos. WO 96/37155, WO 96/37256, WO 96/17648, WO 97/03718, WO 98/11937, WO 98/00193, WO 97/48440, WO 97/48441, WO 97/48442, WO 98/00193, WO 99/64580, WO 98/28037, WO 98/29298, and WO 98/29365; all incorporated herein by reference in their entirety.

The disclosed systems and apparatus employ piercing elements of various shapes and sizes to pierce the outermost layer (i.e., the stratum corneum) of the skin. The piercing elements disclosed in these references generally extend perpendicularly from a thin, flat member, such as a pad or sheet. The piercing elements in some of these devices are extremely small, some having a microprojection length of only about 25-400 microns and a microprojection thickness of only about 5-50 microns. These tiny piercing/cutting elements make correspondingly small microslits/microcuts in the stratum corneum for enhancing transdermal agent delivery therethrough.

The disclosed systems further typically include a reservoir for holding the agent and also a delivery system to transfer the agent from the reservoir through the stratum corneum, such as by hollow tines of the device itself. One example of such a device is disclosed in WO 93/17754, which has a liquid agent reservoir. The reservoir must, however, be pressurized to force the liquid agent through the tiny tubular elements and into the skin. Disadvantages of such devices include the added complication and expense for adding a pressurizable liquid reservoir and complications due to the presence of a pressure-driven delivery system.

As disclosed in U.S. patent application No. 10/045,842, which is fully incorporated by reference herein, it is also possible to have the active agent that is to be delivered coated on the microprojections instead of contained in a physical reservoir. This eliminates the necessity of a separate physical reservoir and developing an agent formulation or composition specifically for the reservoir.

A drawback of the coated microprojection systems is, however, that the maximum amount of delivered active agent, and in particular, immunologically active agents, is limited, since the ability of the microprojections (and arrays thereof) to penetrate the stratum corneum is reduced as the coating thickness increases. A further drawback is that the coated microprojection systems that are presently available are limited to delivery of one active agent.

It would therefore be desirable to provide an apparatus and method for transdermal delivery of multiple biologically active agents, particularly, immunologically active agents via coated microprojections.

It would also be desirable to provide a convenient method for simultaneous administration of several vaccines that may be incompatible from a physicochemical standpoint.

It is therefore an object of the present invention to provide an apparatus and method for simultaneous transdermal delivery of multiple immunologically active agents that substantially reduces or eliminates the drawbacks and disadvantages associated with prior art immunologically active agent delivery methods and systems.

It is another object of the present invention to provide an apparatus and method for substantially simultaneous transdermal delivery of multiple vaccines that includes a microprojection array having a plurality of array regions coated with different biocompatible coatings; each coating including a different vaccine.

It is another object of the present invention to provide an apparatus and method for substantially simultaneous transdermal delivery of multiple vaccines that includes a microprojection array having a plurality of microprojections, at least two of the plurality of microprojections being coated with a different biocompatible coating having a different vaccine or a vaccine and an adjuvant disposed therein.

### SUMMARY OF THE INVENTION

In accordance with the above objects and those that will be mentioned and will become apparent below, the apparatus and method for transdermally delivering multiple immunologically active agents in accordance with one embodiment of the invention generally comprises a delivery system having a microprojection array that includes a plurality of microprojections that are adapted to pierce through the stratum corneum into the underlying epidermis layer, or epidermis and dermis layers, the microprojection array having a plurality of array regions, at least two of the array regions having a different biocompatible coating disposed thereon, wherein at least one of the array region coatings includes at least one immunologically active agent.

In one embodiment, the biocompatible coating on each array region includes different immunologically active agent.

In another embodiment, the biocompatible coating in a first array region includes an immunologically active agent and the biocompatible coating in a second array region includes an adjuvant.

Preferably, the immunologically active agent comprises an antigenic agent or vaccine selected from the group consisting of viruses and bacteria, protein-based vaccines, polysaccharide-based vaccine, nucleic acid-based vaccines, and immune response augmenting adjuvants.

Suitable antigenic agents include, without limitation, antigens in the form of proteins, polysaccharide conjugates, oligosaccharides, and lipoproteins. These subunit vaccines in include Bordetella pertussis (recombinant PT accince—acellular), Clostridium tetani (purified, recombinant), Corynebacterium diphtheriae (purified, recombinant), Cytomegalovirus (glycoprotein subunit), Group A streptococcus (glycoprotein subunit, glycoconjugate Group A polysaccharide with tetanus toxoid, M protein/peptides linked to toxing subunit carriers, M protein, multivalent type-specific epitopes, cysteine protease, C5a peptidase), Hepatitis B virus (recombinant Pre S1, Pre-S2, S, recombinant core protein), Hepatitis C virus (recombinant—expressed surface proteins and epitopes), Human papillomavirus (Capsid protein, TA-GN recombinant protein L2 and E7 [from HPV-6], MEDI-501 recombinant VLP L1 from HPV-11, Quadrivalent recombinant BLP L1 [from HPV-6], HPV-11, HPV-16, and HPV-18, LAMP-E7 [from HPV-16]), Legionella pneumophila (purified bacterial survace protein), Neisseria meningitides (glycoconjugate with tetanus toxoid), Pseudomonas aeruginosa (synthetic peptides), Rubella virus (synthetic peptide), Streptococcus pneumoniae (glycoconjugate [1, 4, 5, 6B, 9N, 14, 18C, 19V, 23F] conjugated to meningococcal B OMP, glycoconjugate [4, 6B, 9V, 14, 18C, 19F, 23F] conjugated to CRM197, glycoconjugate [1, 4, 5, 6B, 9V, 14, 18C, 19F, 23F] conjugated to CRM1970, Treponema pallidum (surface lipoproteins), Varicella zoster virus (subunit, glycoproteins), and Vibrio cholerae (conjugate lipopolysaccharide).

Whole virus or bacteria include, without limitation, weakened or killed viruses, such as cytomegalo virus, hepatitis B virus, hepatitis C virus, human papillomavirus, rubella virus, and varicella zoster, weakened or killed bacteria, such as bordetella pertussis, clostridium tetani, corynebacterium diphtheriae, group A streptococcus, legionella pneumophila, neisseria meningitdis, pseudomonas aeruginosa, streptococcus pneumoniae, treponema pallidum, and vibrio cholerae, and mixtures thereof.

Additional commercially available vaccines, which contain antigenic agents, include, without limitation, flu vaccines, including influenza flu vaccine, Lyme disease vaccine, rabies vaccine, measles vaccine, mumps vaccine, rubella vaccine, pertussis vaccine, tetanus vaccine, typhoid vaccine, rhinovirus vaccine, hemophilus influenza B vaccine, polio vaccine, pneumococal vaccine, menningococcal vaccine, RSU vaccine, herpes vaccine, HIV vaccine, chicken pox vaccine, small pox vaccine, hepatitis vaccine (including types A,B and D) and diphtheria vaccine.

Vaccines comprising nucleic acids include, without limitation, single-stranded and double-stranded nucleic acids, such as, for example, supercoiled plasmid DNA; linear plasmid DNA; cosmids; bacterial artificial chromosomes (BACs); yeast artificial chromosomes (YACs); mammalian artificial chromosomes; and RNA molecules, such as, for example, mRNA. The nucleic acid can also be coupled with a proteinaceous agent or can include one or more chemical modifications, such as, for example, phosphorothioate moieties.

Suitable immune response augmenting adjuvants which, together with the vaccine antigen, can comprise the vaccine include aluminum phosphate gel; aluminum hydroxide; algal glucan: β-glucan; cholera toxin B subunit; CRL1005: ABA block polymer with mean values of x=8 and y=205; gamma inulin: linear (unbranched) β-D(2->1) polyfructofuranoxyl-α-D-glucose; Gerbu adjuvant: N-acetylglucosamine-(β1-4)-N-acetylmuramyl-L-alanyl-D-glutamine (GMDP), dimethyl dioctadecylammonium chloride (DDA), zinc L-proline salt complex (Zn-Pro-8); Imiquimod (1-(2-methypropyl)-1H-imidazo[4,5-c]quinolin-4-amine; ImmTher™: N-acetylglucoaminyl-N-acetylmuramyl-L-Ala-D-isoGlu-L-Ala-glycerol dipalmitate; MTP-PE liposomes: C₅₉H₁₀₈N₆O₁₉PNa-3H₂O (MTP); Murametide: Nac-Mur-L-Ala-D-Gln-OCH₃; Pleuran: β-glucan; QS-21; S-28463: 4-amino-a, a-dimethyl-1H-imidazo[4,5-c]quinoline-1-ethanol; salvo peptide: VQGEESNDK.HCl (IL-1β163-171 peptide); and threonyl-MDP (Termurtide™): N-acetyl muramyl-L-threonyl-D-isoglutamine, and interleukine 18, IL-2 IL-12, IL-15, Adjuvants also include DNA oligonucleotides, such as, for example, CpG containing oligonucleotides. In addition, nucleic acid sequences encoding for immuno-regulatory lymphokines such as IL-18, IL-2 IL-12, IL-15, IL4, IL10, gamma interferon, and NF kappa B regulatory signaling proteins can be used.

The immune response augmenting adjuvant can be formulated separately or with the vaccine antigen.

In one embodiment of the invention, the microprojection array has a microprojection density of at least approximately 10 microprojections/cm², preferably, of at least approximately 100 microprojections/cm², and more preferably, in the range of at least approximately 200-3000 microprojections/cm².

Preferably, the microprojections have a projection length less than 145 microns, more preferably, in the range of approximately 50-145 microns, and even more preferably, in the range of approximately 70-140 microns.

In one embodiment, the microprojection array is constructed out of stainless steel, titanium, nickel titanium alloys, or similar biocompatible materials.

In an alternative embodiment, the microprojection array is constructed out of a non-conductive material, such as a polymer. Alternatively, the microprojection array can be coated with a non-conductive material, such as Parylene®.

In one embodiment of the invention, each biocompatible coating preferably has a thickness less than 100 microns. In a preferred embodiment, each biocompatible coating has a thickness in the range of approximately 2-50 microns.

The coating formulation(s) applied to the microprojection array regions to form the solid biocompatible coatings of the invention can comprise an aqueous or non-aqueous formulation, which, in at least one embodiment, includes at least one immunologically active agent. In a preferred embodiment, the coating formulations comprise aqueous formulations.

In one embodiment of the invention, each coating formulation includes at least one surfactant, which can be zwitterionic, amphoteric, cationic, anionic, or nonionic, Suitable surfactants include, without limitation, sodium lauroamphoacetate, sodium dodecyl sulfate (SDS), cetylpyridinium chloride (CPC), dodecyltrimethyl ammonium chloride (TMAC), benzalkonium, chloride, polysorbates, such as Tween 20 and Tween 80, other sorbitan derivatives, such as sorbitan laurate, and alkoxylated alcohols, such as laureth-4.

In a further embodiment of the invention, at least one coating formulation, preferably, each coating formulation includes at least one polymeric material or polymer that has amphiphilic properties. Suitable polymers having amphiphilic properties include, without limitation, dextrans, hydroxyethyl starch (HES), cellulose derivatives, such as hydroxyethylcellulose (HEC), hydroxypropylmethylcellulose (HPMC), hydroxypropycellulose (HPC), methylcellulose (MC), hydroxyethylmethylcellulose (HEMC), or ethylhydroxy-ethylcellulose (EHEC), as well as pluronics.

In one embodiment of the invention, the concentration of the polymer presenting amphiphilic properties in the coating formulation(s) is preferably in the range of approximately 0.001-70 wt. %, more preferably, in the range of approximately 0.01-50 wt. %, even more preferably, in the range of approximately 0.03-30 wt. % of the coating formulation.

In another embodiment, at least one coating formulation, preferably, each coating formulation includes at least one hydrophilic polymer selected from the following group: poly(vinyl alcohol), poly(ethylene oxide), poly(2-hydroxyethyl-methacrylate), poly(n-vinyl pyrolidone), polyethylene glycol and mixtures thereof, and like polymers.

In a preferred embodiment, the concentration of the hydrophilic polymer in the coating formulation(s) is preferably in the range of approximately 0.001-90 wt. %, more preferably, in the range of approximately 0.01-20 wt. %, even more preferably, in the range of approximately 0.03-10 wt. % of the coating formulation.

In another embodiment of the invention, at least one coating formulation, preferably, each coating formulation includes a biocompatible carrier, which can comprise, without limitation, human albumin, bioengineered human albumin, polyglutamic acid, polyaspartic acid, polyhistidine, pentosan polysulfate, polyamino acids, sucrose, trehalose, melezitose, raffinose and stachyose.

Preferably, the concentration of the biocompatible carrier in the coating formulation(s) is preferably in the range of approximately 0.001-90%, more preferably, in the range of approximately 2-70 wt. %, even more preferably, in the range of approximately 5-50 wt. % of the coating formulation.

In a further embodiment, at least one coating formulation, preferably, each coating formulation includes a stabilizing agent, which can comprise, without limitation, a non-reducing sugar, a polysaccharide, a reducing sugar, or a DNase inhibitor.

In another embodiment, at least one coating formulation, preferably, each coating formulation includes a vasoconstrictor, which can comprise, without limitation, amidephrine, cafaminol, cyclopentamine, deoxyepinephrine, epinephrine, felypressin, indanazoline, metizoline, midodrine, naphazoline, nordefrin, octodrine, ornipressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin, xylometazoline and the mixtures thereof. The most preferred vasoconstrictors include epinephrine, naphazoline, tetrahydrozoline indanazoline, metizoline, tramazoline, tymazoline, oxymetazoline and xylometazoline.

The concentration of the vasoconstrictor, if employed, is preferably in the range of approximately 0.1 wt. % to 10 wt. % of the coating formulation(s).

In yet another embodiment of the invention, at least one coating formulation, preferably, each coating formulation includes at least one “pathway patency modulator”, which can comprise, without limitation, osmotic agents (e.g., sodium chloride), zwitterionic compounds (e.g., amino acids), and anti-inflammatory agents, such as betamethasone 21-phosphate disodium salt, triamcinolone acetonide 21-disodium phosphate, hydrocortamate hydrochloride, hydrocortisone 21-phosphate disodium salt, methylprednisolone 21-phosphate disodium salt, methylprednisolone 21-succinaate sodium salt, paramethasone disodium phosphate and prednisolone 21-succinate sodium salt, and anticoagulants, such as citric acid, citrate salts (e.g., sodium citrate), dextrin sulfate sodium, aspirin and EDTA.

Preferably, each coating formulation of the invention has a viscosity less than approximately 5 poise, more preferably, in the range of approximately 0.3-2.0 poise.

In accordance with one embodiment of the invention, the method for simultaneously delivering multiple immunologically active agents comprises the following steps: (i) providing a microprojection array having a plurality of microprojections, the microprojection array having a plurality of array regions, (ii) coating at least a first microprojection in a first array region with a first biocompatible coating having a first immunologically active agent, (iii) coating at least a second microprojection in a second array region with a second biocompatible coating having a second immunologically active agent, and (iv) applying the coated microprojection array to the skin of a subject.

In accordance with a further embodiment of the invention, the method for delivering multiple immunologically active agents comprises the following steps: (i) providing a microprojection array having a plurality of microprojections, the microprojection array having at least first and second array regions (ii) coating the first array region with a first biocompatible coating, the first biocompatible coating including an immunologically active agent, (iii) coating the second array region with a second biocompatible coating, the second biocompatible coating including an immune response augmenting adjuvant, and (iv) applying the coated microprojection array to the skin of a subject.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 is a perspective view of a portion of one embodiment of a microprojection array, according to the invention;

FIG. 2 is a perspective view of the microprojection array shown in FIG. 1 having a biocompatible coating deposited on the microprojections;

FIG. 3 is a sectioned side view of a microprojection array having an adhesive backing, according to the invention;

FIG. 4 is a perspective view of a portion of another embodiment of a microprojection array, according to the invention;

FIGS. 5 through 7 are schematic illustrations of several embodiments of microprojection arrays having various microprojection array regions and patterns thereof, according to the invention;

FIG. 8 is a sectioned side view of a retainer having a microprojection member disposed therein, according to the invention;

FIG. 9 is a perspective view of the retainer shown in FIG. 8; and

FIG. 10 is a perspective view of an applicator and the retainer shown in FIG. 8.

### DETAILED DESCRIPTION OF THE INVENTION

Before describing the present invention in detail, it is to be understood that this invention is not limited to particularly exemplified materials, formulations, methods or structures as such may, of course, vary. Thus, although a number of materials and methods similar or equivalent to those described herein can be used in the practice of the present invention, the preferred materials and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

Finally, as used in this specification and the appended claims, the singular forms “a, “an” and “the” include plural referents unless the content clearly dictates otherwise. Thus, for example, reference to “an immunologically active agent” includes two or more such agents; reference to “a microprojection” includes two or more such microprojections and the like.

### DEFINITIONS

The term “transdermal”, as used herein, means the delivery of an agent into and/or through the skin for local or systemic therapy.

The term “transdermal flux”, as used herein, means the rate of transdermal delivery.

The term “co-delivering”, as used herein, means that a supplemental agent(s) is administered transdermally either before the agent is delivered, before and during transdermal flux of the agent, during transdermal flux of the agent, during and after transdermal flux of the agent, and/or after transdermal flux of the agent. Additionally, two or more immunologically active agents may be formulated in one biocompatible coating of the invention, resulting in co-delivery of different immunologically active agents from one array region.

The term “biologically active agent”, as used herein, refers to a composition of matter or mixture containing an active agent or drug, which is pharmacologically effective when administered in a therapeutically effective amount. Examples of such active agents include, without limitation, small molecular weight compounds, polypeptides, proteins, oligonucleotides, nucleic acids and polysaccharides.

The term “immunologically active agent”, as used herein, refers to a composition of matter or mixture containing an antigenic agent and/or a “vaccine” derived from any source, which is capable of triggering a beneficial immune response when administered in an immunologically effective amount. Examples of immunologically active agents include, without limitation, viruses and bacteria, protein-based vaccines, polysaccharide-based vaccine, and nucleic acid-based vaccines.

Suitable immunologically active agents include, without limitation, antigens in the form of proteins, polysaccharide conjugates, oligosaccharides, and lipoproteins. These subunit vaccines in include Bordetella pertussis (recombinant PT accince—acellular), Clostridium tetani (purified, recombinant), Corynebacterium diphtheriae (purified, recombinant), Cytomegalovirus (glycoprotein subunit), Group A streptococcus (glycoprotein subunit, glycoconjugate Group A polysaccharide with tetanus toxoid, M protein/peptides linked to toxing subunit carriers, M protein, multivalent type-specific epitopes, cysteine protease, C5a peptidase), Hepatitis B virus (recombinant Pre S1, Pre-S2, S, recombinant core protein), Hepatitis C virus (recombinant—expressed surface proteins and epitopes), Human papillomavirus (Capsid protein, TA-GN recombinant protein L2 and E7 [from HPV-6], MEDI-501 recombinant VLP L1 from HPV-11, Quadrivalent recombinant BLP L1 [from HPV-6], HPV-1, HPV-16, and HPV-18, LAMP-E7[from HPV-16]), Legionella pneumophila (purified bacterial surface protein), Neisseria meningitides (glycoconjugate with tetanus toxoid), Pseudomonas aeruginosa (synthetic peptides), Rubella virus (synthetic peptide), Streptococcus pneumoniae (glycoconjugate [1, 4, 5, 6B, 9N, 14, 18C, 19V, 23F] conjugated to meningococcal B OMP, glycoconjugate [4, 6B, 9V, 14, 18C, 19F, 23F] conjugated to CRM197, glycoconjugate [1, 4, 5, 6B, 9V, 14, 18C, 19F, 23F] conjugated to CRM1970, Treponema pallidum (surface lipoproteins), Varicella zoster virus (subunit, glycoproteins), and Vibrio cholerae (conjugate lipopolysaccharide).

Whole virus or bacteria include, without limitation, weakened or killed viruses, such as cytomegalo virus, hepatitis B virus, hepatitis C virus, human papillomavirus, rubella virus, and varicella zoster, weakened or killed bacteria, such as bordetella pertussis, clostridium tetani, corynebacterium diphtheriae, group A streptococcus, legionella pneumophila, neisseria meningitis, pseudomonas aeruginosa, streptococcus pneumoniae, treponema pallidum, and vibrio cholerae, and mixtures thereof.

A number of commercially available vaccines, which contain antigenic agents also have utility with the present invention, include, without limitation, flu vaccines, Lyme disease vaccine, rabies vaccine, measles vaccine, mumps vaccine, chicken pox vaccine, small pox vaccine, hepatitis vaccine, pertussis vaccine, and diphtheria vaccine.

Vaccines comprising nucleic acids that can also be delivered according to the methods of the invention, include, without limitation, single-stranded and double-stranded nucleic acids, such as, for example, supercoiled plasmid DNA; linear plasmid DNA; cosmids; bacterial artificial chromosomes (BACs); yeast artificial chromosomes (YACs); mammalian artificial chromosomes; and RNA molecules, such as, for example, mRNA. The size of the nucleic acid can be up to thousands of kilobases. The nucleic acid can also be coupled with a proteinaceous agent or can include one or more chemical modifications, such as, for example, phosphorothioate moieties.

Suitable immune response augmenting adjuvants which, together with the vaccine antigen, can comprise the vaccine include, without limitation, aluminum phosphate gel; aluminum hydroxide; algal glucan: β-glucan; cholera toxin B subunit; CRL1005: ABA block polymer with mean values of x=8 and y=205; gamma inulin: linear (unbranched) β-D(2->1) polyfructofuranoxyl-α-D-glucose; Gerbu adjuvant: N-acetylglucosamine-(β 1-4)-N-acetylmuramyl-L-alanyl-D-glutamine (GMDP), dimethyl dioctadecylammonium chloride (DDA), zinc L-proline salt complex (Zn-Pro-8); Imiquimod (1-(2-methypropyl)-1H-imidazo[4,5-c]quinolin-4-amine; ImmTher™: N-acetylglucoaminyl-N-acetylmuramyl-L-Ala-D-isoGlu-L-Ala-glycerol dipalmitate; MTP-PE liposomes: C₅₉H₁₀₈N₆O₁₉PNa-3H₂O (MTP); Murametide: Nac-Mur-L-Ala-D-Gln-OCH₃; Pleuran: β-glucan; QS-21; S-28463: 4-amino-a, a-dimethyl-1H-imidazo[4,5-c]quinoline-1-ethanol; salvo peptide: VQGEESNDK.HCl (IL-1β 163-171 peptide); and threonyl-MDP (Termurtide™): N-acetyl muramyl-L-threonyl-D-isoglutamine, and interleukine 18, IL-2 IL-12, IL-15, Adjuvants also include DNA oligonucleotides, such as, for example, CpG containing oligonucleotides. In addition, nucleic acid sequences encoding for immuno-regulatory lymphokines such as IL-18, IL-2 IL-12, IL-15, IL-4, IL10, gamma interferon, and NF kappa B regulatory signaling proteins can be used.

The term “biologically effective amount” or “biologically effective rate”, as used herein, refers to the amount or rate of the immunologically active agent needed to stimulate or initiate the desired immunologic, often beneficial result. The amount of the immunologically active agent employed in the coatings of the invention will be that amount necessary to deliver an amount of the immunologically active agent needed to achieve the desired immunological result. In practice, this will vary widely depending upon the particular immunologically active agent being delivered, the site of delivery, and the dissolution and release kinetics for delivery of the immunologically active agent into skin tissues.

As will be appreciated by one having ordinary skill in the art, the dose of the immunologically active agent that is delivered from each array region can also be varied or manipulated by altering the microprojection array (or patch) size, density, etc.

The term “coating formulation”, as used herein, is meant to mean and include a freely flowing composition or mixture that is employed to coat the microprojections and/or array regions.

The terms “biocompatible coating” and “solid coating”, as used herein, are meant to mean and include a “coating formulation” in a substantially solid state.

The term “microprojections”, as used herein, refers to piercing elements that are adapted to pierce or cut through the stratum corneum into the underlying epidermis layer, or epidermis and dermis layers, of the skin of a living animal, particularly a mammal and more particularly a human.

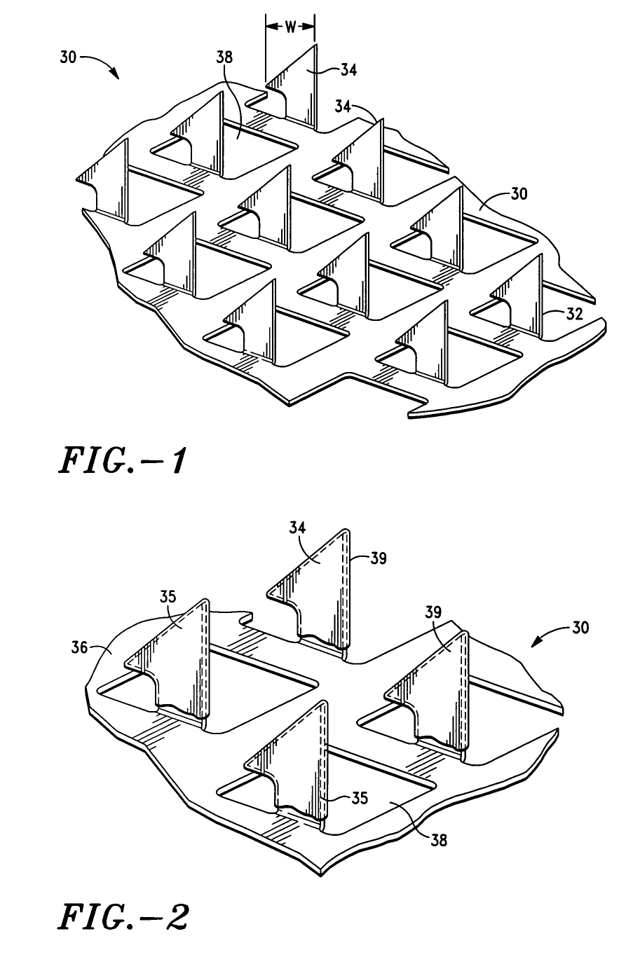

In one embodiment of the invention, the piercing elements have a projection length less than 1000 microns. In a further embodiment, the piercing elements have a projection length of less than 500 microns, more preferably, less than 250 microns. The microprojections further have a width (designated “W” in FIG. 1) in the range of approximately 25-500 microns and a thickness in the range of approximately 10-100 microns. The microprojections may be formed in different shapes, such as needles, blades, pins, punches, and combinations thereof.

In a further embodiment adapted to minimize bleeding and irritation, the microprojections preferably have a projection length less than 145 microns, more preferably, in the range of approximately 50-145 microns, and even more preferably, in the range of approximately 70-140 microns.

The terms “microprojection array” and “microprojection member”, as used herein, generally connotes a plurality of microprojections arranged in an array for piercing the stratum corneum. The microprojection array can be formed by etching or punching a plurality of microprojections from a thin sheet and folding or bending the microprojections out of the plane of the sheet to form a configuration, such as that shown in FIG. 1. The microprojection array can also be formed in other known manners, such as by forming one or more strips having microprojections along an edge of each of the strip(s) as disclosed in U.S. Pat. No. 6,050,988, which is hereby incorporated by reference in its entirety.

As indicated above, the present invention comprises an apparatus and method for transdermal delivery of multiple immunologically active agents that includes a delivery system having a microprojection array that includes a plurality of microprojections that are adapted to pierce through the stratum corneum into the underlying epidermis layer, or epidermis and dermis layers, the microprojection array having a plurality of array regions, at least two of the array regions having a different biocompatible coating disposed thereon, wherein at least one of the coatings includes a least one immunologically active agent.

In one embodiment of the invention, at least the first array region coating includes a first immunologically active agent and at least the second array region coating includes an immune response augmenting adjuvant.

In another embodiment, the first array region coating includes a first immunologically active agent and the second array region coating includes a second immunologically active agent.

In a preferred embodiment, the first and second immunologically active agents are different.

According to the invention, upon piercing the stratum corneum layer of the skin, the biocompatible coating in each array region is dissolved by body fluid (intracellular fluids and extracellular fluids such as interstitial fluid) and the immunologically active agent or agents are released into the skin (i.e., bolus delivery) for systemic therapy.

As will be appreciated by one having ordinary skill in the art, the present invention thus provides a convenient and highly efficient method for administration of multiple vaccines, whether compatible or incompatible from a physicochemical standpoint.

According to the invention, the kinetics of each coating dissolution and release will depend on many factors, including the nature of the immunologically active agent(s), the coating process, the coating thickness and the coating composition (e.g., the presence of coating formulation additives). Depending on the release kinetics profile, it may be necessary to maintain the coated microprojections in piercing relation with the skin for extended periods of time. This can be accomplished by anchoring the microprojection member to the skin using adhesives (or adhesive layers) or by using anchored microprojections, such as shown in FIG. 4 and described in WO 97/48440, which is incorporated by reference herein in its entirety.

Referring now to FIGS. 1 and 2, there is shown one embodiment of a microprojection member (or patch) 30 for use with the present invention. As illustrated in FIG. 1, the microprojection member 30 includes a microprojection array 32 having a plurality of microprojections 34. The microprojections 34 preferably extend at substantially a 90° angle from the sheet 36, which in the noted embodiment includes openings 38 (see FIG. 2).

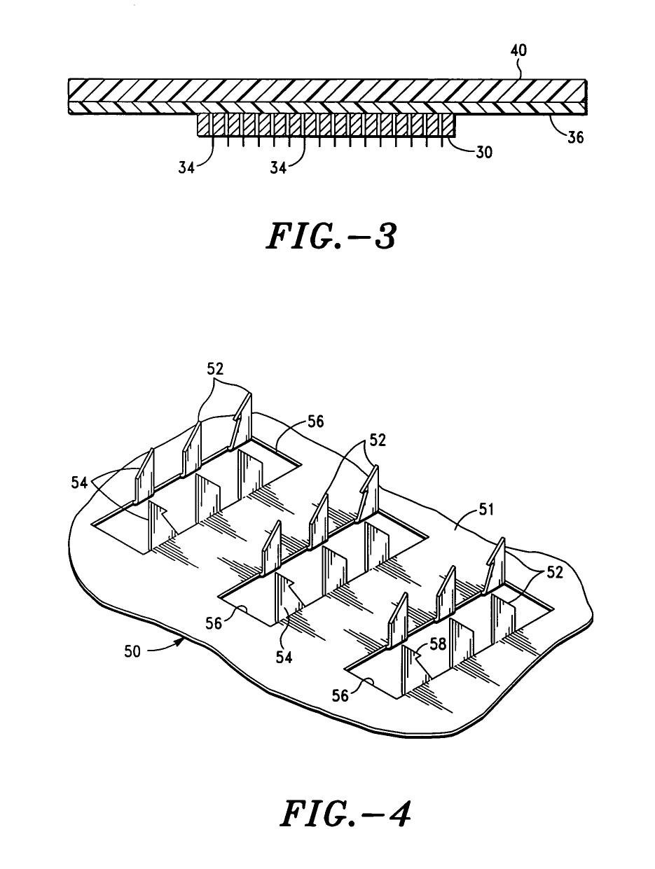

According to the invention, the sheet 36 may be incorporated into a delivery patch, including a backing 40 for the sheet 36, and may additionally include an adhesive strip (not shown) for adhering the patch to the skin (see FIG. 3). In this embodiment, the microprojections 34 are formed by etching or punching a plurality of microprojections 34 from a thin metal sheet 36 and bending the microprojections 34 out of the plane of the sheet 36.

In one embodiment of the invention, the microprojection array 32 has a microprojection density of at least approximately 10 microprojections/cm², preferably, at least approximately 100 microprojections/cm², more preferably, in the range of at least approximately 200-3000 microprojections/cm². Also preferably, the number of openings per unit area through which the agent passes is at least approximately 10 openings/cm² and less than about 3000 openings/cm².

As indicated, the microprojections 34 preferably have a projection length less than 1000 microns. In one embodiment, the microprojections 34 have a projection length of less than 500 microns, more preferably, less than 250 microns. The microprojections 34 also preferably have a width in the range of approximately 25-500 microns and thickness in the range of approximately 10-100 microns. In a currently preferred embodiment, the microprojections have a length in the range of approximately 50-145 microns, and more preferably, in the range of approximately 70-140 microns.

Referring now to FIG. 4, there is shown another embodiment of a microprojection member 50 that can be employed within the scope of the invention. The microprojection member 50 similarly includes a microprojection array 52 having a plurality of microprojections 54. The microprojections 54 preferably extend at substantially a 90° angle from the sheet 51, which similarly includes openings 56.

As illustrated in FIG. 4, several of the microprojections 54 include a retention member or anchor 58 disposed proximate the leading edge. As indicated above, the retention member 58 facilitates adherence of the microprojection member 50 to the subject's skin.

The microprojection members (e.g., 30, 50) and/or arrays can be manufactured from various metals, such as stainless steel, titanium, nickel titanium alloys, or similar biocompatible materials. Preferably, the microprojection member is manufactured out of titanium.

According to the invention, the microprojection members and arrays can also be constructed out of a non-conductive material, such as a polymer. Alternatively, the microprojection member and/or array can be coated with a non-conductive material, such as Parylene®, or a hydrophobic material, such as Teflon®, silicon or other low energy material. The noted hydrophobic materials and associated base (e.g., photoreist) layers are set forth in U.S. Provisional Application No. 60/484,142, which is incorporated by reference herein.

Microprojection members and arrays that can be employed with the present invention include, but are not limited to, the members disclosed in U.S. Pat. Nos. 6,083,196, 6,050,988 and 6,091,975, and U.S. patent Pub. No. 2002/0016562, which are incorporated by reference herein in their entirety.

Other microprojection members and arrays that can be employed with the present invention include members formed by etching silicon using silicon chip etching techniques or by molding plastic using etched micro-molds, such as the members disclosed U.S. Pat. No. 5,879,326, which is incorporated by reference herein in its entirety.

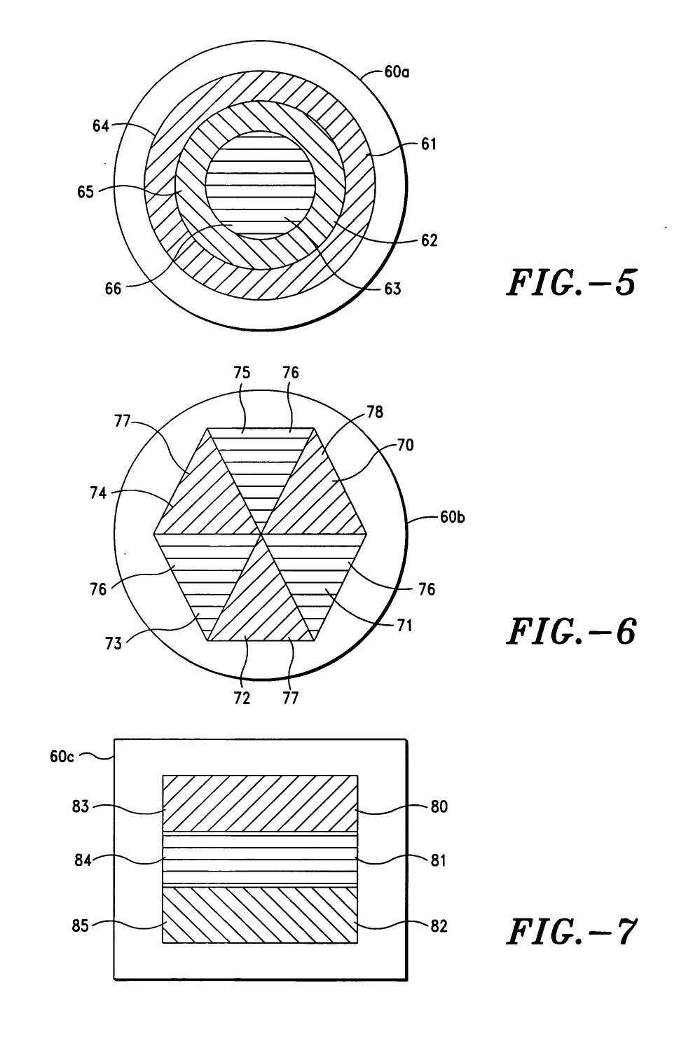

Referring now to FIGS. 5-7, there are shown various microprojection arrays 60a, 60b, 60c having various array region patterns. It is to be understood that the arrays 60a, 60b, 60c and array patterns associated therewith are merely exemplary patterns and thus should not be construed as limiting the scope of the invention in any way. Indeed, as will be appreciated by one having ordinary skill in the art, the microprojection arrays and patterns can comprise various shapes, sizes and configurations. The array regions can also be joined (i.e., physically connected) or spaced apart. Further, the number and location of the vaccine containing-biocompatible coatings can also vary to facilitate delivery of different compatible and/or incompatible vaccines and the desired dosage thereof.

Referring now to FIG. 5, the noted microprojection array 60a includes three substantially circular and distinct array regions 61, 62, 63. As stated, each array region 61, 62, 63 can have a substantially similar or dissimilar size and, hence, area.

According to the invention, each array region 61, 62, 63 includes a biocompatible coating 64, 65, 66 having at least one immunologically active agent disposed therein. In the noted embodiment, each biocompatible coating 64, 65, 66 in each array region 61, 62, 63 contains a different immunologically active agent.

In an alternative embodiment, one immunologically active agent is contained in two array regions, e.g., regions 61 and 63, and a different immunologically active agent is contained in the remaining array region, e.g., region 62.

Referring now to FIG. 6, there is shown a further microprojection array 60b having a hexagonal shaped pattern that is preferably divided into six array regions 70 through 75. According to the invention, the array regions 70-75 can similarly have substantially similar or dissimilar shapes and sizes.

In the noted embodiment, array regions 71, 73 and 75 include a first biocompatible coating 76 containing a first immunologically active agent; array regions 72 and 74 include a second biocompatible coating 77 containing a second immunologically active agent; and array region 70 includes a third biocompatible coating 78 containing a third immunologically active agent.

As stated, the number and location of the different coatings and, hence, vaccines disposed therein can be varied to accommodate the delivery of a desired number of vaccines and/or dosages thereof. By way of example, in one alternative embodiment, each array region 70-75 contains a different coating having a different immunologically agent disposed therein.

Referring now to FIG. 7, there is shown yet another embodiment of a microprojection array 60c. As illustrated in FIG. 7, the microprojection array 60c has a substantially rectangular shape and includes a substantially rectangular array pattern.

In the illustrated embodiment, the array pattern includes three linear array regions 80, 81, 82. According to the invention, the array regions 80, 81, 82 can similarly be substantially similar or dissimilar in shape.

As illustrated in FIG. 7, each array region 80, 81, 82 includes a different biocompatible coating 83, 84, 85 having at least one different immunologically active agent disposed therein.

Similarly, the number of linear regions, and number and location of the different coatings and, hence, vaccines disposed therein can be varied to accommodate the delivery of a desired number of vaccines and/or dosages thereof. By way of example, in an alternative embodiment, the array includes five linear regions, each region containing a different coating having a different immunologically active agent disposed therein.

Referring now to FIG. 2, there is shown a portion of a microprojection array 30 having microprojections 34 coated with a biocompatible coating 35. According to the invention, the coating 35 can partially or completely cover each microprojection 34. For example, the coating 35 can be in a dry pattern coating on the microprojections 34. The coating 35 can also be applied before or after the microprojections 34 are formed.

According to the invention, the coating 35 in each array region can be applied to the microprojections 34 by a variety of known methods. Preferably, the coating is only applied to those portions the microprojection member 30 or microprojections 34 that pierce the skin (e.g., tips 39).

One such coating method comprises dip-coating. Dip-coating can be described as a means to coat the microprojections by partially or totally immersing the microprojections 34 into a coating solution. By use of a partial immersion technique, it is possible to limit the coating 35 to only the tips 39 of the microprojections 34.

A further coating method comprises roller coating, which employs a roller coating mechanism that similarly limits the coating 35 to the tips 39 of the microprojections 34. The roller coating method is disclosed in U.S. application Ser. No. 10/099,604 (Pub. No. 2002/0132054), which is incorporated by reference herein in its entirety. As discussed in detail in the noted application, the disclosed roller coating method provides a smooth coating that is not easily dislodged from the microprojections 34 during skin piercing.

According to the invention, the microprojections 34 can further include means adapted to receive and/or enhance the volume of the coating 35, such as apertures (not shown), grooves (not shown), surface irregularities (not shown) or similar modifications, wherein the means provides increased surface area upon which a greater amount of coating can be deposited.

A further coating method that can be employed within the scope of the present invention comprises spray coating. According to the invention, spray coating can encompass formation of an aerosol suspension of the coating composition. In one embodiment, an aerosol suspension having a droplet size of about 10 to 200 picoliters is sprayed onto the microprojections 10 and then dried.

Pattern coating can also be employed to coat the microprojections 34. The pattern coating can be applied using a dispensing system for positioning the deposited liquid onto the microprojection surface. The quantity of the deposited liquid is preferably in the range of 0.1 to 20 nanoliters/microprojection. Examples of suitable precision-metered liquid dispensers are disclosed in U.S. Pat. Nos. 5,916,524; 5,743,960; 5,741,554; and 5,738,728; which are fully incorporated by reference herein.

Microprojection coating formulations or solutions can also be applied using ink jet technology using known solenoid valve dispensers, optional fluid motive means and positioning means which is generally controlled by use of an electric field. Other liquid dispensing technology from the printing industry or similar liquid dispensing technology known in the art can be used for applying the pattern coating of this invention.

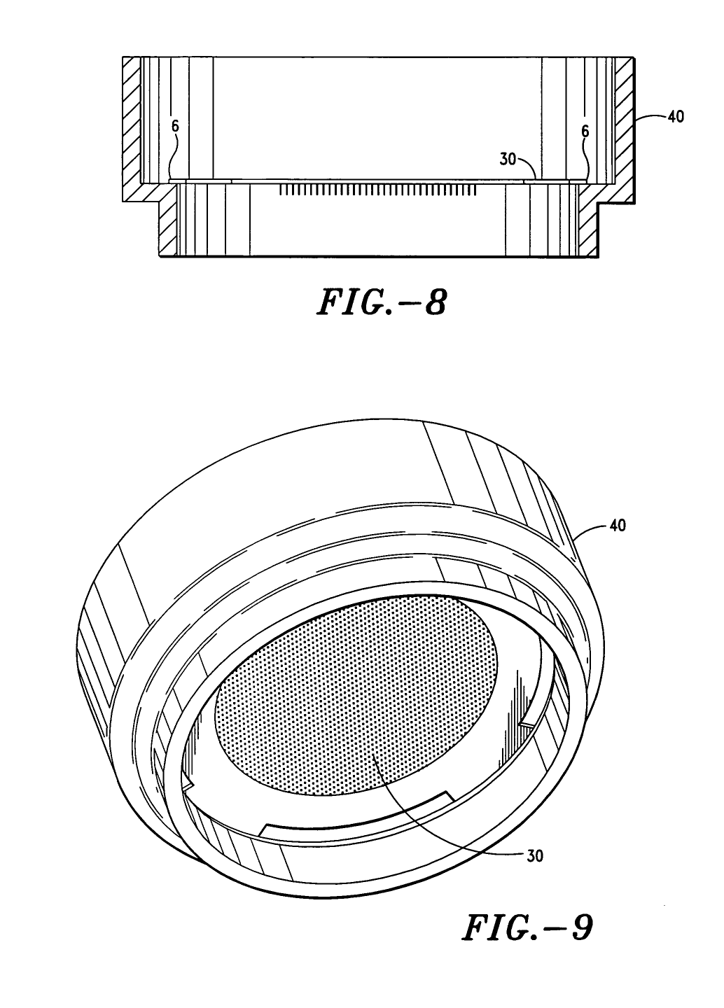

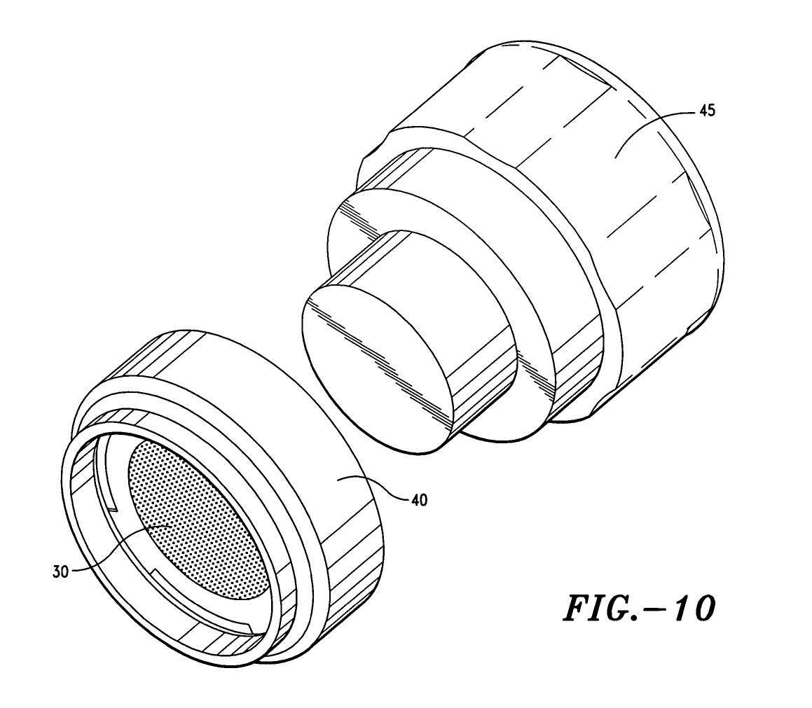

Referring now to FIGS. 8 and 9, for storage and application, the microprojection array 30 is preferably suspended in a retainer ring 40 by adhesive tabs 6, as described in detail in Co-Pending U.S. application Ser. No. 09/976,762 (Pub. No. 2002/0091357), which is incorporated by reference herein in its entirety.

After placement of the microprojection member 30 in the retainer ring 40, the microprojection member 30 is applied to the patient's skin. Preferably, the microprojection member 30 is applied to the skin using an impact applicator 45, such as shown in FIG. 10 and disclosed in Co-Pending U.S. application Ser. No. 09/976,798, which is incorporated by reference herein in its entirety.

As indicated, in a preferred embodiment of the invention, the coating formulations applied to the microprojection array 32 to form the solid coatings comprise an aqueous formulations. In an alternative embodiment, the coating formulations comprise a non-aqueous formulation. According to the invention, each immunologically active agent can be dissolved within a biocompatible carrier or suspended within the carrier.

As indicated, in a preferred embodiment of the invention, the immunologically active agent comprises a vaccine (or antigenic agent) selected from the group consisting of viruses and bacteria, protein-based vaccines, polysaccharide-based vaccine, and nucleic acid-based vaccines.

Suitable antigenic agents include, without limitation, antigens in the form of proteins, polysaccharide conjugates, oligosaccharides, and lipoproteins. These subunit vaccines in include Bordetella pertussis (recombinant PT accince—acellular), Clostridium tetani (purified, recombinant), Corynebacterium diphtheriae (purified, recombinant), Cytomegalovirus (glycoprotein subunit), Group A streptococcus (glycoprotein subunit, glycoconjugate Group A polysaccharide with tetanus toxoid, M protein/peptides linked to toxing subunit carriers, M protein, multivalent type-specific epitopes, cysteine protease, C5a peptidase), Hepatitis B virus (recombinant Pre S1, Pre-S2, S, recombinant core protein), Hepatitis C virus (recombinant—expressed surface proteins and epitopes), Human papillomavirus (Capsid protein, TA-GN recombinant protein L2 and E7[from HPV-6], MEDI-501 recombinant VLP L1 from HPV-11, Quadrivalent recombinant BLP L1[from HPV-6], HPV-11, HPV-16, and HPV-18, LAMP-E7[from HPV-16]), Legionella pneumophila (purified bacterial surface protein), Neisseria meningitides (glycoconjugate with tetanus toxoid), Pseudomonas aeruginosa (synthetic peptides), Rubella virus (synthetic peptide), Streptococcus pneumoniae (glycoconjugate [1, 4, 5, 6B, 9N, 14, 18C, 19V, 23F] conjugated to meningococcal B OMP, glycoconjugate [4, 6B, 9V, 14, 18C, 19F, 23F] conjugated to CRM197, glycoconjugate [1, 4, 5, 6B, 9V, 14, 18C, 19F, 23F] conjugated to CRM 1970, Treponema pallidum (surface lipoproteins), Varicella zoster virus (subunit, glycoproteins), and Vibrio cholerae (conjugate lipopolysaccharide).

Whole virus or bacteria include, without limitation, weakened or killed viruses, such as cytomegalo virus, hepatitis B virus, hepatitis C virus, human papillomavirus, rubella virus, and varicella zoster, weakened or killed bacteria, such as bordetella pertussis, clostridium tetani, corynebacterium diphtheriae, group A streptococcus, legionella pneumophila, neisseria meningitis, pseudomonas aeruginosa, streptococcus pneumoniae, treponema pallidum, and vibrio cholerae, and mixtures thereof.

Additional commercially available vaccines, which contain antigenic agents, include, without limitation, flu vaccines, including influenza flu vaccine, Lyme disease vaccine, rabies vaccine, measles vaccine, mumps vaccine, rubella vaccine, pertussis vaccine, tetanus vaccine, typhoid vaccine, rhinovirus vaccine, hemophilus influenza B, polio vaccine, pneumococal vaccine, menningococcal vaccine, RSU vaccine, herpes vaccine, HIV vaccine, chicken pox vaccine, small pox vaccine, hepatitis vaccine (including types A,B and D) and diphtheria vaccine.

Vaccines comprising nucleic acids include, without limitation, single-stranded and double-stranded nucleic acids, such as, for example, supercoiled plasmid DNA; linear plasmid DNA; cosmids; bacterial artificial chromosomes (BACs); yeast artificial chromosomes (YACs); mammalian artificial chromosomes; and RNA molecules, such as, for example, mRNA. The size of the nucleic acid can be up to thousands of kilobases. In addition, in certain embodiments of the invention, the nucleic acid can be coupled with a proteinaceous agent or can include one or more chemical modifications, such as, for example, phosphorothioate moieties. The encoding sequence of the nucleic acid comprises the sequence of the antigen against which the immune response is desired. In addition, in the case of DNA, promoter and polyadenylation sequences are also incorporated in the vaccine construct. The antigen that can be encoded include all antigenic components of infectious diseases, pathogens, as well as cancer antigens. The nucleic acids thus find application, for example, in the fields of infectious diseases, cancers, allergies, autoimmune, and inflammatory diseases.

Suitable immune response augmenting adjuvants which, together with the vaccine antigen, can comprise the vaccine include, without limitation, aluminum phosphate gel; aluminum hydroxide; algal glucan: β-glucan; cholera toxin B subunit; CRL1005: ABA block polymer with mean values of x=8 and y=205; gamma inulin: linear (unbranched) β-D(2->1) polyfructofuranoxyl-α-D-glucose; Gerbu adjuvant: N-acetylglucosamine-(β 1-4)-N-acetylmuramyl-L-alanyl-D-glutamine (GMDP), dimethyl dioctadecylammonium chloride (DDA), zinc L-proline salt complex (Zn-Pro-8); Imiquimod (1-(2-methypropyl)-1H-imidazo[4,5-c]quinolin-4-amine; ImmTher™: N-acetylglucoaminyl-N-acetylmuramyl-L-Ala-D-isoGlu-L-Ala-glycerol dipalmitate; MTP-PE liposomes: C₅₉H₁₀₈N₆O₁₉PNa-3H₂O (MTP); Murametide: Nac-Mur-L-Ala-D-Gln-OCH₃; Pleuran: β-glucan; QS-21; S28463: 4-amino-a, a-dimethyl-1H-imidazo[4,5-c]quinoline-1-ethanol; salvo peptide: VQGEESNDK.HCl (IL-1β 163-171 peptide); and threonyl-MDP (Termurtide™): N-acetyl muramyl-L-threonyl-D-isoglutamine, and interleukine 18, IL-2 IL-12, IL-15, Adjuvants also include DNA oligonucleotides, such as, for example, CpG containing oligonucleotides. In addition, nucleic acid sequences encoding for immuno-regulatory lymphokines such as IL-18, IL-2 IL-12, IL-15, IL-4, IL10, gamma interferon, and NF kappa B regulatory signaling proteins can be used.

According to the invention, each coating formulation can include at least one wetting agent. Suitable wetting agents include surfactants and polymers that present amphiphilic properties.

Thus, in one embodiment of the invention, at least one coating formulation, preferably, each coating formulation includes at least one surfactant. According to the invention, the surfactant(s) can be zwitterionic, amphoteric, cationic, anionic, or nonionic. Examples of suitable surfactants include, sodium lauroamphoacetate, sodium dodecyl sulfate (SDS), cetylpyridinium chloride (CPC), dodecyltrimethyl ammonium chloride (TMAC), benzalkonium, chloride, polysorbates such as Tween 20 and Tween 80, other sorbitan derivatives such as sorbitan laurate, and alkoxylated alcohols such as laureth-4. Most preferred surfactants include Tween 20, Tween 80, and SDS.

In a further embodiment of the invention, at least one coating formulation, preferably, each coating formulation includes at least one polymeric material or polymer that has amphiphilic properties. Examples of the noted polymers include, without limitation, cellulose derivatives, such as hydroxyethylcellulose (HEC), hydroxyl-propylmethylcellulose (HPMC), hydroxyl-propylcellulose (HPC), methylcellulose (MC), hydroxyethylmethylcellulose (HEMC), or ethylhydroxyethylcellulose (EHEC), as well as pluronics.

In one embodiment of the invention, the concentration of the polymer presenting amphiphilic properties is preferably in the range of approximately 0.01-20 wt. %, more preferably, in the range of approximately 0.03-10 wt. % of the coating formulation. Even more preferably, the concentration of the polymer is in the range of approximately 0.1-5 wt. % of the coating formulation.

As will be appreciated by one having ordinary skill in the art, the noted wetting agents can be used separately or in combinations.

According to the invention, at least one coating formulation, preferably, each coating formulation can further include a hydrophilic polymer. Preferably the hydrophilic polymer is selected from the following group: dextrans, hydroxyethyl starch (HES), poly(vinyl alcohol), poly(ethylene oxide), poly(2-hydroxyethylmethacrylate), poly(n-vinyl pyrolidone), polyethylene glycol and mixtures thereof, and like polymers. As is well known in the art, the noted polymers increase viscosity.

The concentration of the hydrophilic polymer in the coating formulation(s) is preferably in the range of approximately 0.01-50 wt. %, more preferably, in the range of approximately 0.03-30 wt. % of the coating formulation. Even more preferably, the concentration of the hydrophilic polymer is in the range of approximately 0.1-20 wt. % of the coating formulation.

According to the invention, at least one coating formulation, preferably, each coating formulation includes a biocompatible carrier, such as those disclosed in Co-Pending U.S. application Ser. No. 10/127,108, which is incorporated by reference herein in its entirety. Examples of biocompatible carriers include human albumin, bioengineered human albumin, polyglutamic acid, polyaspartic acid, polyhistidine, pentosan polysulfate, polyamino acids, sucrose, trehalose, melezitose, raffinose and stachyose.

The concentration of the biocompatible carrier in the coating formulation(s) is preferably in the range of approximately 2-70 wt. %, more preferably, in the range of approximately 5-50 wt. % of the coating formulation. Even more preferably, the concentration of the carrier is in the range of approximately 10-40 wt. % of the coating formulation.

According to the invention, at least one coating formulation, preferably, each coating formulation can further include a vasoconstrictor, such as those disclosed in Co-Pending U.S. application Ser. No. 10/674,626, which is incorporated by reference herein in their entirety. As set forth in the noted Co-Pending Application, the vasoconstrictor is used to control bleeding during and after application on the microprojection member. Preferred vasoconstrictors include, but are not limited to, amidephrine, cafaminol, cyclopentamine, deoxyepinephrine, epinephrine, felypressin, indanazoline, metizoline, midodrine, naphazoline, nordefrin, octodrine, ornipressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin, xylometazoline and the mixtures thereof. The most preferred vasoconstrictors include epinephrine, naphazoline, tetrahydrozoline indanazoline, metizoline, tramazoline, tymazoline, oxymetazoline and xylometazoline.

The concentration of the vasoconstrictor, if employed, is preferably in the range of approximately 0.1 wt. % to 10 wt. % of the coating formulation.

In yet another embodiment of the invention, at least one coating formulation, preferably, each coating formulation includes at least one “pathway patency modulator”, such as those disclosed in Co-Pending U.S. application Ser. No. 09/950,436, which is incorporated by reference herein in its entirety. As set forth in the noted Co-Pending Application, the pathway patency modulators prevent or diminish the skin's natural healing processes thereby preventing the closure of the pathways or microslits formed in the stratum corneum by the microprojection member array. Examples of pathway patency modulators include, without limitation, osmotic agents (e.g., sodium chloride), and zwitterionic compounds (e.g., amino acids).

The term “pathway patency modulator”, as defined in the Co-Pending Application, further includes anti-inflammatory agents, such as betamethasone 21-phosphate disodium salt, triamcinolone acetonide 21-disodium phosphate, hydrocortamate hydrochloride, hydrocortisone 21-phosphate disodium salt, methylprednisolone 21-phosphate disodium salt, methylprednisolone 21-succinaate sodium salt, paramethasone disodium phosphate and prednisolone 21-succinate sodium salt, and anticoagulants, such as citric acid, citrate salts (e.g., sodium citrate), dextrin sulfate sodium, aspirin and EDTA.

According to the invention, each coating formulation can also include a non-aqueous solvent, such as ethanol, chloroform, ether, propylene glycol, polyethylene glycol and the like, dyes, pigments, inert fillers, permeation enhancers, excipients, and other conventional components of pharmaceutical products or transdermal devices known in the art.

Other known formulation adjuvants can also be added to the coating formulations as long as they do not adversely affect the necessary solubility and viscosity characteristics of the coating formulations and the physical integrity of the dried coating.

Preferably, each coating formulation has a viscosity less than approximately 5 poise in order to effectively coat each microprojection 10. More preferably, each coating formulation has a viscosity in the range of approximately 0.3-2.0 poise.

According to the invention, the median coating thickness of each array region is preferably less than 100 microns, more preferably less than 50 microns. Even more preferably, the coating thickness is in the range of approximately 2-30 microns.

The desired coating thickness is dependent upon several factors, including the required dosage and, hence, coating thickness necessary to deliver the dosage, the density of the microprojections per unit area of the sheet, the viscosity and concentration of the coating formulation employed at each array region and the coating method chosen.

In all cases, after the coating formulations have has been applied, each coating formulation can be dried on the microprojections by various means. In one embodiment of the invention, the coated microprojection array is air-dried in ambient room conditions. In another embodiment, the coated microprojection array is vacuum-dried. In yet another embodiment, the coated microprojection array is air-dried and vacuum-dried thereafter.

Various temperatures and humidity levels can also be employed to dry the coating formulations on the microprojections. The coated microprojection array can thus be heated, lyophilized, freeze dried or subjected to similar techniques to remove the water from the coatings.

In accordance with one embodiment of the invention, the method for simultaneously delivering multiple immunologically active agents comprises the following steps: (i) providing a microprojection array having a plurality of microprojections, the microprojection array having a plurality of array regions, (ii) coating at least a first microprojection in a first array region with a first biocompatible coating having a first immunologically active agent, (iii) coating at least a second microprojection in a second array region with a second biocompatible coating having a second immunologically active agent, and (iv) applying the coated microprojection array to the skin of a subject.

As will be appreciated by one having ordinary skill in the art, the present invention is not limited solely to delivery of multiple vaccines. Indeed, the invention can readily be employed to facilitate delivery of multiple allergens for desensitation procedures or allergy testing.

Further, vaccination against some pathogens would require immunization with multiple isotypes that may not be compatible, e.g., Pseudomonas with 23 isotypes. The invention can thus be readily employed to facilitate such vaccination.

Also, co-delivery of immune-enhancing adjuvants may be necessary to increase the immunogenicity of a vaccine to ensure seropropection. Thus, in alternative embodiments of the invention, the microprojection array can include (i) at least a first array region being coated with a first biocompatible coating containing a vaccine and at least a second array region being coated with a second biocompatible coating containing an adjuvant or (ii) at least a first array region being coated with a first biocompatible coating containing a first vaccine, at least a second array region being coated with a second biocompatible coating containing a second vaccine and at least a third array region being coated with a third biocompatible coating containing an adjuvant or (iii) at least a first array region being coated with a first biocompatible coating containing a plurality of vaccines and at least a second array region being coated with a second biocompatible coating containing an adjuvant.

Accordingly, in accordance with a further embodiment of the invention, the method for delivering multiple immunologically active agents comprises the following steps: (i) providing a microprojection array having a plurality of microprojections, the microprojection array having first and second array regions (ii) coating the first array region with a first biocompatible coating, the first biocompatible coating including an immunologically active agent, (iii) coating the second array region with a second biocompatible coating, the second biocompatible coating including an immune response augmenting adjuvant, and (iv) applying the coated microprojection array to the skin of a subject.

Without departing from the spirit and scope of this invention, one of ordinary skill can make various changes and modifications to the invention to adapt it to various usages and conditions. As such, these changes and modifications are properly, equitably, and intended to be, within the full range of equivalence of the above described embodiments.

1. A system for transdermally delivering multiple immunologically active agents, comprising a microprojection array having a plurality of stratum corneum-piercing microprojections, said microprojection array having at least first and second array regions, said first array region having a first biocompatible coating disposed thereon, said second array region having a second biocompatible coating disposed thereon, wherein said first biocompatible coating includes at least one immunologically active agent.

2. The system of claim 1, wherein said second biocompatible coating includes an immune response augmenting adjuvant.

3. The system of claim 1, wherein said immunologically active agent is selected from the group consisting of viruses, bacteria, protein-based vaccines, polysaccharide-based vaccine, and nucleic acid-based vaccines.

4. The system of claim 1, wherein said immunologically active agent is selected from the group consisting of viruses, weakened viruses, killed viruses, bacteria, weakened bacteria, killed bacteria, protein-based vaccines, polysaccharide-based vaccine, nucleic acid-based vaccines, proteins, polysaccharide conjugates, oligosaccharides, lipoproteins, Bordetella pertussis (recombinant PT vaccine—acellular), Clostridium tetani (purified, recombinant), Corynebacterium diphtheriae (purified, recombinant), Cytomegalovirus (glycoprotein subunit), Group A streptococcus (glycoprotein subunit, glycoconjugate Group A polysaccharide with tetanus toxoid, M protein/peptides linked to toxing subunit carriers, M protein, multivalent type-specific epitopes, cysteine protease, C5a peptidase), Hepatitis B virus (recombinant Pre S1, Pre-S2, S, recombinant core protein), Hepatitis C virus (recombinant—expressed surface proteins and epitopes), Human papillomavirus (Capsid protein, TA-GN recombinant protein L2 and E7[from HPV-6], MEDI-501 recombinant VLP L1 from HPV-11, Quadrivalent recombinant BLP L1 [from HPV-6], HPV-11, HPV-16, and HPV-18, LAMP-E7[from HPV-16]), Legionella pneumophila (purified bacterial survace protein), Neisseria meningitides (glycoconjugate with tetanus toxoid), Pseudomonas aeruginosa (synthetic peptides), Rubella virus (synthetic peptide), Streptococcus pneumoniae (glycoconjugate [1, 4, 5, 6B, 9N, 14, 18C, 19V, 23F] conjugated to meningococcal B OMP, glycoconjugate [4, 6B, 9V, 14, 18C, 19F, 23F] conjugated to CRM197, glycoconjugate [1, 4, 5, 6B, 9V, 14, 18C, 19F, 23F] conjugated to CRM1970, Treponema pallidum (surface lipoproteins), Varicella zoster virus (subunit, glycoproteins), Vibrio cholerae (conjugate lipopolysaccharide), cytomegalo virus, hepatitis B virus, hepatitis C virus, human papillomavirus, rubella virus, varicella zoster, bordetella pertussis, clostridium tetani, corynebacterium diphtheriae, group A streptococcus, legionella pneumophila, neisseria meningitdis, pseudomonas aeruginosa, streptococcus pneumoniae, treponema pallidum, vibrio cholerae, flu vaccines, Lyme disease vaccines, rabies vaccines, measles vaccines, mumps vaccines, chicken pox vaccines, small pox vaccines, hepatitis vaccines, pertussis vaccines, diphtheria vaccines, nucleic acids, single-stranded nucleic acids, double-stranded nucleic acids, supercoiled plasmid DNA, linear plasmid DNA, cosmids, bacterial artificial chromosomes (BACs), yeast artificial chromosomes (YACs), mammalian artificial chromosomes, RNA molecules, and mRNA.

5. The system of claim 1, wherein said immunologically active agent includes an immune response augmenting adjuvant selected from the group consisting of aluminum phosphate gel, aluminum hydroxide, alpha glucan, β-glucan, cholera toxin B subunit, CRL1005, ABA block polymer with mean values of x=8 and y=205, gamma inulin, linear (unbranched) β-D(2->1) polyfructofuranoxyl-α-D-glucose, Gerbu adjuvan, N-acetylglucosamine-(β 1-4)-N-acetylmuramyl-L-alanyl-D-glutamine (GMDP), dimethyl dioctadecylammonium chloride (DDA), zinc L-proline salt complex (Zn-Pro-8), Imiquimod (1-(2-methypropyl)-1H-imidazo[4,5-c]quinolin-4-amine, ImmTher™, N-acetylglucoaminyl-N-acetylmuramyl-L-Ala-D-isoGlu-L-Ala-glycerol dipalmitate, MTP-PE liposomes, C₅₉H₁₀₈N₆O₁₉PNa-3H₂O (MTP), Murametide, Nac-Mur-L-Ala-D-Gln-OCH₃, Pleuran, QS-21; S-28463, 4-amino-a, a-dimethyl-1H-imidazo[4,5-c]quinoline-1-ethanol, sclavo peptide, VQGEESNDK.HCl (IL-1β 163-171 peptide), threonyl-MDP (Termurtide™), N-acetyl muramyl-L-threonyl-D-isoglutamine, interleukine 18 (IL-18), IL-2 IL-12, IL-15, IL-4, IL-10, DNA oligonucleotides, CpG containing oligonucleotides, gamma interferon, and NF kappa B regulatory signaling proteins.

6. The system of claim 2, wherein said immune response augmenting adjuvant is selected from the group consisting of aluminum phosphate gel, aluminum hydroxide, alpha glucan, β-glucan, cholera toxin B subunit, CRL1005, ABA block polymer with mean values of x=8 and y=205, gamma inulin, linear (unbranched) β-D(2->1) polyfructofuranoxyl-α-D-glucose, Gerbu adjuvan, N-acetylglucosamine-(β 1-4)-N-acetylmuramyl-L-alanyl-D-glutamine (GMDP), dimethyl dioctadecylammonium chloride (DDA), zinc L-proline salt complex (Zn-Pro-8), Imiquimod (1-(2-methypropyl)-1H-imidazo[4,5-c]quinolin-4-amine, ImmTher™, N-acetylglucoaminyl-N-acetylmuramyl-L-Ala-D-isoGlu-L-Ala-glycerol dipalmitate, MTP-PE liposomes, C₅₉H₁₀₈N₆O₁₉PNa-3H₂O (MTP), Murametide, Nac-Mur-L-Ala-D-Gln-OCH₃, Pleuran, QS-21; S-28463, 4-amino-a, a-dimethyl-1H-imidazo[4,5-c]quinoline-1-ethanol, sclavo peptide, VQGEESNDK.HCl (IL-1β 163-171 peptide), threonyl-MDP (Termurtide™M), N-acetyl muramyl-L-threonyl-D-isoglutamine, interleukine 18 0(IL-18), IL-2 IL-12, IL-15, IL4, IL-10, DNA oligonucleotides, CpG containing oligonucleotides, gamma interferon, and NF kappa B regulatory signaling proteins.

7. The system of claim 1, wherein said microprojection member has a microprojection density of at least approximately 100 microprojections/cm².

8. The system of claim 7, wherein said microprojection member has a microprojection density in the range of approximately 200-3000 microprojections/cm².

9. The system of claim 1, wherein each of said microprojections has a length less than 1000 microns.

10. The system of claim 9, wherein each of said microprojections has a length in the range of approximately 50-145 microns.

11. The system of claim 1, wherein said first and second biocompatible coatings have a thickness in the range of approximately 2-50 microns.

12. The system of claim 1, wherein said first and second biocompatible coatings are formed from a coating formulation.

13. The system of claim 12, wherein said coating formulation comprises an aqueous formulation.

14. The system of claim 12, wherein said coating formulation includes a surfactant.

15. The system of claim 14, wherein said surfactant is selected from the group consisting of sodium lauroamphoacetate, sodium dodecyl sulfate (SDS), cetylpyridinium chloride (CPC), dodecyltrimethyl ammonium chloride (TMAC), benzalkonium, chloride, polysorbates, such as Tween 20 and Tween 80, sorbitan derivatives, sorbitan laurate, alkoxylated alcohols, and laureth-4.

16. The system of claim 12, wherein said coating formulation includes an amphiphilic polymer.

17. The system of claim 16, wherein said amphiphilic polymer is selected from the group consisting of cellulose derivatives, hydroxyethylcellulose (HEC), hydroxypropyl-methylcellulose (HPMC), hydroxypropycellulose (HPC), methylcellulose (MC), hydroxyethylmethylcellulose (HEMC), ethylhydroxyethylcellulose (EHEC), and pluronics.

18. The system of claim 12, wherein said coating formulation includes a hydrophilic polymer.

19. The system of claim 18, wherein said hydrophilic polymer is selected from the group consisting of poly(vinyl alcohol), poly(ethylene oxide), poly(2-hydroxyethylmethacrylate), poly(n-vinyl pyrolidone), polyethylene glycol and mixtures thereof.

20. The system of claim 12, wherein said coating formulation includes a biocompatible carrier.

21. The system of claim 20, wherein said biocompatible polymer is selected from the group consisting of human albumin, bioengineered human albumin, polyglutamic acid, polyaspartic acid, polyhistidine, pentosan polysulfate, polyamino acids, sucrose, trehalose, melezitose, raffinose and stachyose.

22. The system of claim 12, wherein said coating formulation includes a stabilizing agent selected from the group consisting of a non-reducing sugar, a polysaccharide, a reducing sugar, and a DNase inhibitor.

23. The system of claim 12, wherein said coating formulation includes a vasoconstrictor.

24. The system of claim 23, wherein said vasoconstrictor is selected from the group consisting of epinephrine, naphazoline, tetrahydrozoline indanazoline, metizoline, tramazoline, tymazoline, oxymetazoline, xylometazoline, amidephrine, cafaminol, cyclopentamine, deoxyepinephrine, epinephrine, felypressin, indanazoline, metizoline, midodrine, naphazoline, nordefrin, octodrine, ornipressin, oxymethazoline, phenylephrine, phenylethanolamine, phenylpropanolamine, propylhexedrine, pseudoephedrine, tetrahydrozoline, tramazoline, tuaminoheptane, tymazoline, vasopressin and xylometazoline.

25. The system of claim 12, wherein said coating formulation includes a pathway patency modulator.

26. The system of claim 25, wherein said pathway patency modulator is selected from the group consisting of osmotic agents, sodium chloride, zwitterionic compounds, amino acids, anti-inflammatory agents, betamethasone 21-phosphate disodium salt, triamcinolone acetonide 21-disodium phosphate, hydrocortamate hydrochloride, hydrocortisone 21-phosphate disodium salt, methylprednisolone 21-phosphate disodium salt, methylprednisolone 21-succinaate sodium salt, paramethasone disodium phosphate, prednisolone 21-succinate sodium salt, anticoagulants, citric acid, citrate salts, sodium citrate, dextran sulfate sodium, and EDTA.

27. The system of claim 12, wherein said coating formulation has a viscosity less than approximately 5 poise and greater than approximately 0.3 poise.

28. A system for transdermally delivering multiple immunologically active agents, comprising a microprojection array having a plurality of stratum corneum-piercing microprojections, said microprojection array having at least first and second array regions, said first array region having a first biocompatible coating disposed thereon, said first biocompatible coating including a first immunologically active agent, said second array region having a second biocompatible coating disposed thereon, said second biocompatible coating including a second immunologically active agent.

29. The system of claim 28, wherein said first and second immunologically active agents are different.

30. The system of claim 28, wherein said first and second immunologically active agents are selected from the group consisting of viruses, bacteria, protein-based vaccines, polysaccharide-based vaccine, and nucleic acid-based vaccines.

31. The system of claim 28, wherein said first and second immunologically active agents are selected from the group consisting of viruses, weakened viruses, killed viruses, bacteria, weakened bacteria, killed bacteria, protein-based vaccines, polysaccharide-based vaccine, nucleic acid-based vaccines, proteins, polysaccharide conjugates, oligosaccharides, lipoproteins, Bordetella pertussis (recombinant PT vaccine—acellular), Clostridium tetani (purified, recombinant), Corynebacterium diphtheriae (purified, recombinant), Cytomegalovirus (glycoprotein subunit), Group A streptococcus (glycoprotein subunit, glycoconjugate Group A polysaccharide with tetanus toxoid, M protein/peptides linked to toxing subunit carriers, M protein, multivalent type-specific epitopes, cysteine protease, C5a peptidase), Hepatitis B virus (recombinant Pre S1, Pre-S2, S, recombinant core protein), Hepatitis C virus (recombinant—expressed surface proteins and epitopes), Human papillomavirus (Capsid protein, TA-GN recombinant protein L2 and E7[from HPV-6], MEDI-501 recombinant VLP L1 from HPV-11, Quadrivalent recombinant BLP L1 [from HPV-6], HPV-11, HPV-16, and HPV-18, LAMP-E7[from HPV-16]), Legionella pneumophila (purified bacterial survace protein), Neisseria meningitides (glycoconjugate with tetanus toxoid), Pseudomonas aeruginosa (synthetic peptides), Rubella virus (synthetic peptide), Streptococcus pneumoniae (glycoconjugate [1, 4, 5, 6B, 9N, 14, 18C, 19V, 23F] conjugated to meningococcal B OMP, glycoconjugate [4, 6B, 9V, 14, 18C, 19F, 23F] conjugated to CRM197, glycoconjugate [1, 4, 5, 6B, 9V, 14, 18C, 19F, 23F] conjugated to CRM1970, Treponema pallidum (surface lipoproteins), Varicella zoster virus (subunit, glycoproteins), Vibrio cholerae (conjugate lipopolysaccharide), cytomegalo virus, hepatitis B virus, hepatitis C virus, human papillomavirus, rubella virus, varicella zoster, bordetella pertussis, clostridium tetani, corynebacterium diphtheriae, group A streptococcus, legionella pneumophila, neisseria meningitdis, pseudomonas aeruginosa, streptococcus pneumoniae, treponema pallidum, vibrio cholerae, flu vaccines, Lyme disease vaccines, rabies vaccines, measles vaccines, mumps vaccines, chicken pox vaccines, small pox vaccines, hepatitis vaccines, pertussis vaccines, diphtheria vaccines, nucleic acids, single-stranded nucleic acids, double-stranded nucleic acids, supercoiled plasmid DNA, linear plasmid DNA, cosmids, bacterial artificial chromosomes (BACs), yeast artificial chromosomes (YACs), mammalian artificial chromosomes, RNA molecules, and mRNA.

32. The system of claim 28, wherein said first and second immunologically active agents include an immune response augmenting adjuvant selected from the group consisting of aluminum phosphate gel, aluminum hydroxide, alpha glucan, β-glucan, cholera toxin B subunit, CRL1005, ABA block polymer with mean values of x=8 and y=205, gamma inulin, linear (unbranched) β-D(2->1) polyfructofuranoxyl-α-D-glucose, Gerbu adjuvan, N-acetylglucosamine-(β 1-4)-N-acetylmuramyl-L-alanyl-D-glutamine (GMDP), dimethyl dioctadecylammonium chloride (DDA), zinc L-proline salt complex (Zn-Pro-8), Imiquimod (1-(2-methypropyl)-1H-imidazo[4,5-c]quinolin4-amine, ImmTher™, N-acetylglucoaminyl-N-acetylmuramyl-L-Ala-D-isoGlu-L-Ala-glycerol dipalmitate, MTP-PE liposomes, C₅₉H₁₀₈N₆O₁₉PNa-3H₂O (MTP), Murametide, Nac-Mur-L-Ala-D-Gln-OCH₃, Pleuran, QS-21; S-28463, 4-amino-a, a-dimethyl-1H-imidazo[4,5-c]quinoline-1-ethanol, sclavo peptide, VQGEESNDK.HCl (IL-1β 163-171 peptide), threonyl-MDP (Termurtide™), N-acetyl muramyl-L-threonyl-D-isoglutamine, interleukine 18 (IL-18), IL-2 IL-12, IL-15, IL-4, IL-10, DNA oligonucleotides, CpG containing oligonucleotides, gamma interferon, and NF kappa B regulatory signaling proteins.

33. The system of claim 28, wherein said microprojection member has a microprojection density of at least approximately 100 microprojections/cm².

34. The system of claim 28, wherein said microprojection member has a microprojection density in the range of approximately 200-3000 microprojections/cm².

35. The system of claim 28, wherein each of said microprojections has a length in the range of approximately 50-145 microns.

36. A method for transdermally delivering multiple immunologically active agents to a subject, the method comprising the steps of:
providing a microprojection array having a plurality of microprojections, said microprojection array having at least first and second array regions;
coating said first array region with a first biocompatible coating, said first biocompatible coating including at least one immunologically active agent;
coating said second array region with a second biocompatible coating, said second biocompatible coating including an immune response augmenting adjuvant; and
applying said coated microprojection array to the skin of a subject.

37. A method for transdermally delivering multiple immunologically active agents to a subject, the method comprising the steps of:
providing a microprojection array having a plurality of microprojections, said microprojection array having a plurality of array regions;
coating at least a first microprojection in a first array region with a first biocompatible coating having a first immunologically active agent;
coating at least a second microprojection in a second array region with a second biocompatible coating having a second immunologically active agent; and
applying said coated microprojection array to the skin of a subject.

38. The method of claim 37, wherein said first and second immunologically active agents are different.
